(19) United States
(12) Patent Application Publication
(10) Pub. No.: US 2011/0295103 A1
(43) Pub. Date: Dec. 1, 2011

(54) VISUAL STIMULATION PRESENTING APPARATUS, FUNCTIONAL MAGNETIC RESONANCE IMAGING APPARATUS, MAGNETOENCEPHALOGRAPH APPARATUS, AND BRAIN FUNCTION MEASUREMENT METHOD

(75) Inventors: Yoshikatsu Ichimura, Tokyo (JP); Yasuhiro Kawashima, Tokyo (JP)

(73) Assignee: CANON KABUSHIKI KAISHA, Tokyo (JP)

(21) Appl. No.: 13/117,815

(22) Filed: May 27, 2011

(30) Foreign Application Priority Data
May 31, 2010 (JP) 2010-123819

(51) Int. Cl. A61B 5/05

(52) U.S. Cl. 600/409

(57) ABSTRACT

A visual stimulation presentation apparatus including: a specimen holding unit, made of a non-magnetic material, configured to hold multiple specimens of actual objects serving as specimens for presenting a subject with visual stimulation when running an apparatus to measure brain functions of the subject by detecting change in magnetic field; an actuator, made of a non-magnetic material, configured to selectively present multiple specimens of actual objects set in the specimen holding unit, by driving the specimen holding unit; and a control unit configured to control driving of the actuator; is used, whereby presenting time and presenting position can be precisely controlled and the delicate surface state of the objects with a high degree of sense of texture.

### BACKGROUND OF THE INVENTION

1. Field of the Invention

The present invention relates to a visual stimulation presenting apparatus used for presenting visual stimulation to a subject at the time of running an apparatus to perform brain function measurement of the subject by detecting change in magnetic field, and to a functional magnetic resonance imaging apparatus, a magnetoencephalograph apparatus, and a brain function measurement method.

2. Description of the Related Art

Apparatuses for measuring brain function by detecting change in magnetic field are used in various situations in medical practice. Magnetic resonance imaging (MRI) apparatuses and magnetoencephalograph (MEG) apparatuses are representative of apparatuses which measure brain function by detecting change in magnetic field. Of these, magnetic resonance imaging apparatuses are apparatuses which apply a magnetostatic field to the part of the subject to be measured, and further apply a particular high-frequency electromagnetic field, thereby obtaining an image employing the nuclearmagnetic resonance phenomenon occurring thereby.

Also, an MRI having particular functions, called functional MRI, is used in research in the field of medical practice and, in particular, psychology (hereinafter also abbreviated to “fMRI”). With fMRI, the subject is subjected to various types of stimulation relating to visual perception, tactile perception, auditory perception, and so forth, to research the functions of the brain of the subject, and the responses of the brain to the stimulation are measured using MRI.

With measurement by fMRI, multiple types of stimulation are presented to the subject in order in time sequence, and the relative change in brain activity in response to the stimulation is detected and analysis. Accordingly, with a stimulation presenting device used with a functional magnetic resonance imaging apparatus to carry out the fMRI method, multiple types of stimulation need to be prepared and presented to the subject. In particular, with measurement where visual stimulation is applied, in order to present the multiple stimulation in order of time sequence while precisely controlling the presenting time, a technique is often employed wherein multiple images and pictures are electronically generated and presented to the subject by being displayed on a display or projector on the like.

On the other hand, with measurement using the fMRI method to measure the brain function of the subject, extremely minute changes in the magnetic field of the brain of the subject are measured. Accordingly, an fMRI apparatus has to be installed in a magnetic shielding room, into which no equipment including magnetic substances or equipment emitting electromagnetic noise can be introduced. This means that in the event of performing measurement by fMRI while presenting visual stimulation, electronic equipment such as normal displays or projectors cannot be installed in the shielding room, so various contrivances have been made regarding the apparatuses to provide the stimulation to be presented to the subject during measurement by fMRI so that no electromagnetic noise is generated.

Heretofore, a brain function measurement system of the configuration shown in FIG. 10 has been proposed in Japanese Patent Laid-Open No. 2004-160086 as a technique for providing visual stimulation during measurement with fMRI as described above. With this functional magnetic resonance imaging apparatus to carry out fMRI, a projection room for projecting visual stimulation images is installed in a control room where a control apparatus is installed, adjacent to the shielding room where the MRI apparatus proper that has been electromagnetically shielded is installed. A projection picture is projected on a screen or the like within the shielded room via a projection beam opening provided between the shielding room and the control room, thereby presenting the visual stimulation image to the subject.

Also, with an environment adjusting system and the like proposed in Japanese Patent Laid-Open No. 2009-50474, a technique is disclosed for measuring brain activity with a brain function measuring apparatus by fMRI, while presenting environmental elements to the subject that are sensed by the five senses (e.g., visual perception). With this environment adjusting system and the like, a case is disclosed in which target images such as photographs, illustrations, text, and so forth, which have been printed or drawn on panels or paper, as environment elements recognized by the subject, are presented to the subject by the observer by hand.

Also, with a robotic surgery system capable of handling MRI that is proposed in G. S. Fischer, Proceedings of 2008 IEEE International Conference on Robotics and Automation pp. 2489-2495, an actuator which uses compressed air is employed. With this system, a control unit for compressed air is installed at a position around 2.5 m away from the MRI, and a piston-shaped actuator which is installed within the MRI bore and operates with compressed air is driven.

However, the conventional arrangements described above have the following problems. With that proposed in Japanese Patent Laid-Open No. 2004-160086, a projector apparatus or liquid display apparatus for showing pictures or images, is situated at a position removed from the region to be measured (i.e., the cerebrum of the subject) to reduce electromagnetic noise in the fMRI measurement. The proposal involves images and pictures shot with cameras, video cameras, or the like, being electronically subjected to data processing, and displayed on the projector apparatus or liquid display apparatus, which are then provided to the subject through an optical system as visual stimulation.

To begin with, the act of a human observer seeing an object involves the process of some sort of light source existing, an object being illuminated by a light flux emitted from the light source, an object image made up of reflected light from the illumination being cast on the retina of the observer and imaged, and ultimately the brain processing the biosignals from the retina. However, with a case of using a project which shows images by reflecting or transmitting an image off or through a display or screen using self-emitted light to present an image of an object to an observer, it is extremely difficult to completely reproduce the above-described process of light source, object, and reflection. Accordingly, in the event of performing cognitive experiments relating to medical or psychological practice relating to vision, clearly different visual cognition experiment measurement results will be obtained between a case of observing actual objects and a case of observing through a display or projector.

Particularly, it is generally difficult to represent with high quality the sense of texture, gloss, metallic sheen, and so forth, of the surface of objects using a display or projector, so the sense of reality and presence is understood to be lower as compared to a case of observing an actual object. Accordingly, with a technique of presenting the subject with visual stimulation using a projector or the like such as with Japanese Patent Laid-Open No. 2004-160086, the process of controlling the illumination of the actual object so as to present to the subject is absent, so it is difficult to reproduce states and the like the same as with presenting the actual object. As a result, performing measurement by fMRI while presenting the subject with such objects with delicate surface state and a high degree of sense of texture, with a sense of presence, is difficult.

Also, in the event of using an actual object as a specimen to be presented to the subject in measurement by fMRI, the timing of obtaining the MRI functional image when measuring the brain function, and the timing of presenting the specimen for visual stimulation needs to be controlled in a precise manner. Further, in order to present a fine surface state and a high degree of sense of texture of the object, the positional relation between the light source, object, and the observer (more particularly, the eyes thereof) has to be accurately controlled. Accordingly, in order to attempt measurement by fMRI while presenting visual stimulation using actual objects, the time of presenting the multiple specimens for visual stimulation and the place of presentation needs to be precisely controlled.

With the environment adjusting system and the like proposed in Japanese Patent Laid-Open No. 2009-50474, the state of brain activity is measured while presenting the subject with target images such as photographs, illustrations, text, and so forth, which are perceived through the five senses (e.g., visual perception). However, the target images here are presented by human hand, and it is difficult with such a technique to precisely control the presentation time and presenting position of multiple visual stimulation specimens to be presented to the subject.

Also, with the robotic surgery system capable of handling MRI that is proposed in G. S. Fischer, Proceedings of 2008 IEEE International Conference on Robotics and Automation pp. 2489-2495, a control unit for compressed air is installed at a position around 2.5 m away from the MRI, and a piston-shaped actuator which is installed within the MRI bore and operates with compressed air is driven, so the mechanism is complicated. Also the positional control precision has a margin of around ±5 mm, which is not necessarily satisfactory for a highly precise control technology.

### SUMMARY OF THE INVENTION

Aspects of the present invention are directed to a visual stimulation presenting apparatus, a functional magnetic resonance imaging apparatus, a magnetoencephalograph apparatus, and a brain function measurement method, enabling, at the time of running an apparatus to perform brain function measurement of the subject by detecting change in magnetic field, with regard to selectively presenting to a subject multiple specimens for visual stimulation which are made up of actual objects, realization of precise control of presenting time and presenting position, and presenting a fine surface state of and a high degree of sense of texture of the objects.

Aspects of the present invention provide for a visual stimulation presenting apparatus, a functional magnetic resonance imaging apparatus, a magnetoencephalograph apparatus, and a brain function measurement method, as follows.

According to one aspect of the present invention, a visual stimulation presentation apparatus, used in presenting a subject with visual stimulation when running an apparatus to measure brain functions of the subject by detecting change in magnetic field, includes: a specimen holding unit, made of a non-magnetic material, configured to hold multiple specimens of actual objects as specimens for presenting the subject with visual stimulation; an actuator, made of a non-magnetic material, configured to selectively present multiple specimens of actual objects set in the specimen holding unit to the subject, by driving the specimen holding unit; and a control unit configured to control driving of the actuator.

According to another aspect of the present invention, a functional magnetic resonance imaging apparatus includes the above-described visual stimulation presentation apparatus.

According to another aspect of the present invention, a magnetoencephalograph apparatus includes the above-described visual stimulation presentation apparatus.

According to another aspect of the present invention, a brain function measurement method, using a visual stimulation presentation apparatus which selectively presents a subject with a plurality of visual stimulation specimens, to measure brain functions of the subject by detecting change in magnetic field, includes the steps of: selectively presenting the subject with a plurality of specimens which are actual objects, by driving a specimen holding unit made of a non-magnetic material in which specimens which are actual objects to serve as the plurality of visual stimulation specimens are set, by an actuator made of a non-magnetic material; acquiring images in a state in which no sensory stimulation is applied to the brain of the subject, and in a state in which sensory stimulation is applied to the brain of the subject, the states being alternately repeated; and obtaining a brain function image reflecting the state of activity of the brain, by comparing images acquired in the acquiring in a state in which no sensory stimulation is applied to the brain of the subject with images acquired in the acquiring in a state in which sensory stimulation is applied to the brain of the subject.

According to the present invention, with regard to selectively presenting to a subject multiple specimens for visual stimulation which are made up of actual objects, a visual stimulation presenting apparatus, a functional magnetic resonance imaging apparatus, a magnetoencephalograph apparatus, and a brain function measurement method, can precisely control the presentation time and presenting position to realize presentation of a fine surface state of and a high degree of sense of texture of objects at the time of running an apparatus to perform brain function measurement of the subject by detecting change in magnetic field.

Further features of the present invention will become apparent from the following description of exemplary embodiments with reference to the attached drawings.

### BRIEF DESCRIPTION OF THE DRAWINGS

FIG. 1 is a block diagram of a functional magnetic resonance imaging (fMRI) apparatus having a visual stimulation presenting apparatus according to a first embodiment of the present invention.

FIGS. 2A and 2B are enlarged diagrams of a specimen holder configuring a part of the visual stimulation presenting apparatus according to the first embodiment of the present invention.

FIGS. 3A and 3B are diagrams for describing the specimen holder configuring a part of the visual stimulation presenting apparatus used with the first embodiment of the present invention.

FIGS. 4A through 4C are diagrams for describing a sequence relating to visual stimulation provided to a subject and acquisition of a brain function image according to the first embodiment of the present invention.

FIG. 5 is a diagram illustrating a sequence using a technique called block design that is used with common brain function measurement.

FIGS. 6A and 6B are diagrams illustrating the configuration of a visual stimulation presenting apparatus wherein a specimen holder and actuator are connected by a rotational belt, as another configuration example of the ultrasonic motor according to the first embodiment of the present invention.

FIGS. 7A and 7B are diagrams illustrating the configuration of a visual stimulation presenting apparatus using multiple specimen holders and actuators, according to a second embodiment of the present invention.

FIGS. 8A and 8B are diagrams illustrating the configuration of a visual stimulation presenting apparatus using a linear-drive actuator, according to a third embodiment of the present invention.

FIG. 9 is a diagram illustrating the configuration of a magnetoencephalograph apparatus to which has been applied a visual stimulation presenting apparatus according to a fourth embodiment of the present invention, and the configuration of the visual stimulation presenting apparatus used in magnetoencephalography.

FIG. 10 is a diagram illustrating the configuration of a visual stimulation presenting apparatus used for presenting a subject with visual stimulation, used with a magnetic resonance imaging apparatus for measuring brain functions according to a conventional example.

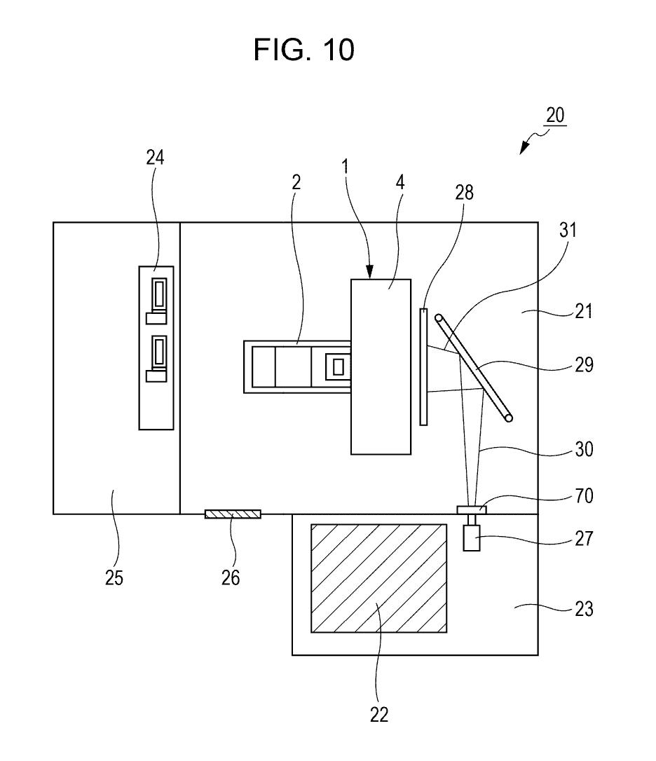

### DESCRIPTION OF THE EMBODIMENTS

A visual stimulation presentation apparatus used for presenting a subject with visual stimulation when running an apparatus to perform brain function measurement of the subject by detecting change in magnetic field is configured so as to present multiple specimens made of actual objects as visual stimulation specimens to be presented to the subject. This is performed by a configuration wherein an image of the object formed by reflected light reflected off of a selected object can be presented within a range directly observable by the subject. Accordingly, deterioration in the quality of the visual information can be suppressed as compared to an arrangement where images obtained by cameras or video cameras or the like, or images generated by simulation or the like are electronically processed and presented on a display or with a projector.

Accordingly, deterioration in the sense of quality and presence of the image can be suppressed, and brain function measurement can be performed while presenting the subject with stimulation having a high sense of quality. Also, a configuration which allows precise control of selectively presenting time and presenting position at the time of presenting the subject with multiple visual stimulation specimens made of actual objects at the time of measuring brain functions enables presenting of delicate surface state and a high degree of sense of texture which the surface of the object has.

Also, while embodiments of the present invention will be described with regard to a functional magnetic resonance imaging apparatus and a magnetoencephalograph apparatus as apparatuses for performing brain function measurement by detecting change in magnetic field, the present invention is not restricted to these.

Next, embodiments of the present invention will be described.

### First Embodiment

In the first embodiment, description will be made regarding an example of installing a visual stimulation presentation apparatus for presenting multiple specimens for visual stimulation that are made up of actual objects to the subject, in a functional magnetic resonance imaging (fMRI) apparatus, and performing brain function measurement. Using a functional magnetic resonance imaging (fMRI) apparatus is preferable, since active portions of the brain in manifestation of brain functions can be imaged with high sensitivity and highly fine images.

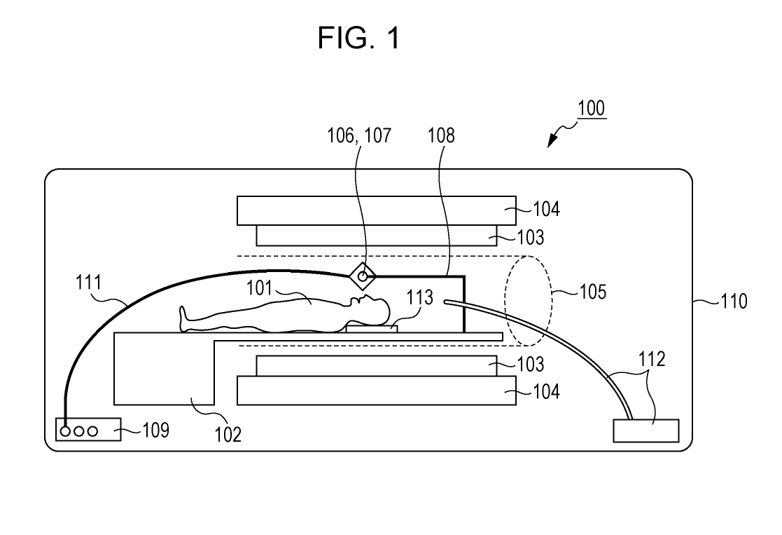

First, in the present embodiment, a configuration example in a case of installing a visual stimulation presentation apparatus for selectively presenting multiple specimens for visual stimulation in a functional magnetic resonance imaging apparatus which performs brain function measurement of a subject will be described with reference to FIG. 1.

As shown in FIG. 1, with the present embodiment, a subject 101 lies on a bed 102 provided to an MRI apparatus 100, and is situated within a measurement portion (bore) 105 where a gradient magnetic field coil 103 and a superconductive magnet 104 are provided. Within the bore, an ultrasonic motor 106 made of a non-magnetic material and a specimen holder 107 made of a non-magnetic material connected to the ultrasonic motor are attached to a specimen presenting apparatus fixing base 108 so as to be situated in front of the eyes of the subject 101.

In order to selectively present multiple specimens made up of actual objects set in the specimen holder 107, an actuator 107 made of a non-magnetic material is configured so as to perform drive the specimen holder 107 by under control of a control unit. Specifically, a controller 109 to control driving of the ultrasonic motor 106 is situated within a magnetic shielding room 110 where the MRI is installed, so as to be maximally distanced from the measurement position within the MRI apparatus proper. The controller 109 is connected to the ultrasonic motor 106 by a electromagnetically shielded control line 111.

The ultrasonic motor 106 is configured so as to be capable of driving under control of the controller 109 regarding the presenting time and presenting position of the multiple visual stimulation presentation specimens made up of actual objects to be selectively presented to the subject. The ultrasonic motor may be one of a configuration driven by a piezoelectric device (electromechanical energy converting device), which is disclosed in many literature documents, such as Japanese Patent Laid-Open No. 3-253272, for example. The casing and shaft of the ultrasonic motor preferably uses a non-magnetic metal, and the bearings are preferably ceramic. Further, as for the non-magnetic metal used for the components such as the casing, a high-resistance material is preferably used to maximally suppress occurrence of eddy current due to magnetic field fluctuation occurring within the MRI, with materials such as titanium and the like being suitably used.

An optical fiber illumination apparatus 112 made of a non-magnetic material for illuminating the specimen on the specimen holder 107 is situated at a place above the head of the subject 101. The optical fiber illumination apparatus 112 has a function of casting illumination light onto the specimen holder 107 from an optional direction (not shown).

Also, an MRI signal detecting coil 113 is disposed behind the head of the subject 101, and detects electromagnetic signals generated by change in cerebral blood flow accompanying neural activity. A surface coil type RF coil is used for the MRI signal detecting coil 113, in order to secure a field of view in front of the eyes of the subject, and to perform high-sensitivity brain function measurement at the visual cortex of the brain, i.e., at the rear of the head of the subject.

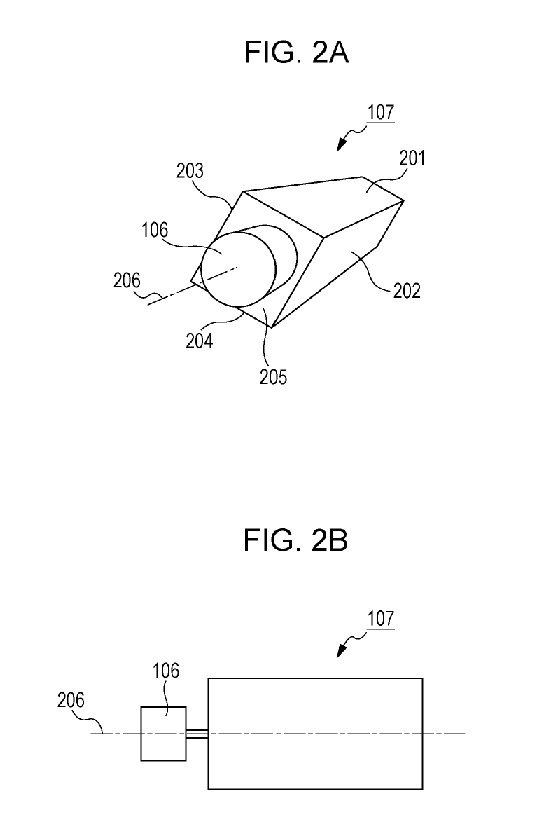

FIG. 2 is an enlarged diagram of the specimen holder 107 making up the visual stimulation presentation apparatus according to the present embodiment. The specimen holder 107 has a rectangular parallelepiped form with specimens to be presented to the subject being placed at four faces 201, 202, 203, and 204, of the rectangular parallelepiped. The ultrasonic motor 106 is attached to one square end face 205 of the rectangular parallelepiped. An axis of rotation 206 of the ultrasonic motor 106 is disposed to the center of the square one end face of the specimen holder 107, such that the specimen holder 107 can rotate on this rotational shaft.

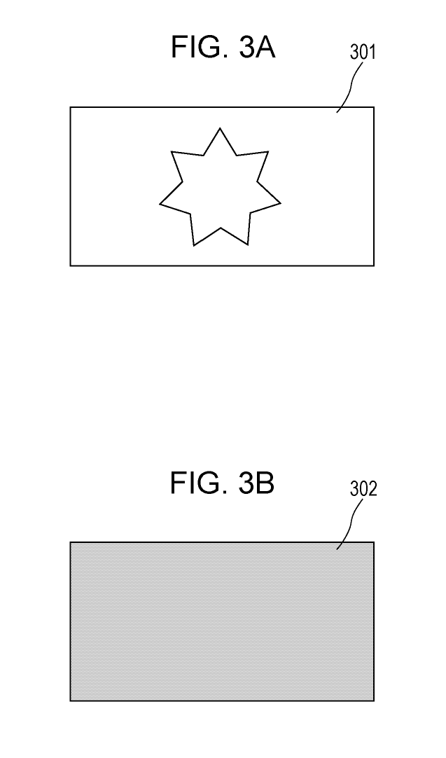

Next, the procedures for brain function measurement using functional magnetic resonance imaging according to the present embodiment will be described with reference to FIGS. 1 and 2. Here, to facilitate description, we will say that measurement is made using two types of drawings printed on paper, such as shown in FIGS. 3A and 3B. FIG. 3A is a specimen 301 on which an arbitrary stimulation drawing has been printed, in order to measure the response of the brain of the subject. On the other hand, the specimen 302 shown in FIG. 3B is a specimen which is a piece of paper the same size as that of the specimen 301 in FIG. 3A, but the entire face is colored uniformly gray. The specimens 301 and 302 are mounted to the faces 201 and 202 of the specimen holder 107. Note that no magnetic substance is included in the specimens 301 and 302, or the ink material or the like used for printing.

Now, the bed 102 is set such that the head of the subject 101 comes to be situated at the MRI measurement position in the MRI bore 105. The specimen holder 107 is set in front of the eyes of the subject 101, such that the subject 101 can observe the specimen 302 mounted to the face 202, and such that the other faces 201, 203, and 204 of the specimen holder 107 (faces 201, 203, and 204; faces 203 and 204 are not shown in FIGS. 3A and 3B) are not visible from the subject 101. Moreover, the face which the subject 101 observes at this time is illuminated by the optical fiber illumination apparatus 112 with a suitable light quantity and incident angle.

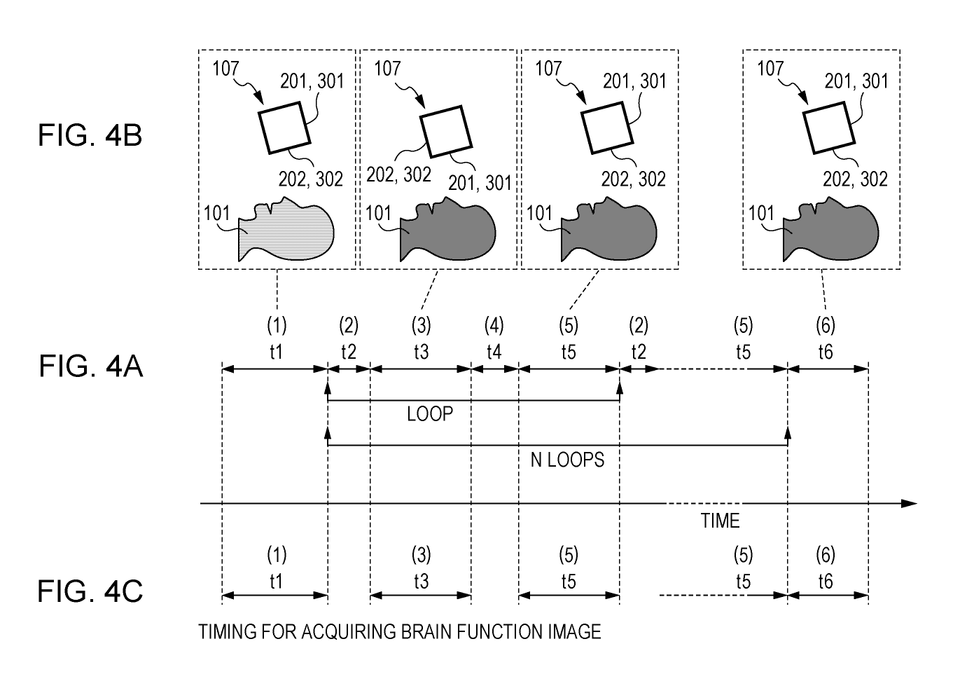

Next, a sequence relating to visual stimulation provided to the subject, and brain function image acquisition, according to the present embodiment, will be described with reference to FIGS. 4A through 4C. First, the visual stimulation sequence provided to the subject will be described with reference to FIGS. 4A and 4B. The subject is subjected to visual stimulation in the following steps (1) through (6). Note that FIG. 4A illustrates the order of the steps and time intervals thereof, and FIG. 4B illustrates the positional relation between the specimen held by the specimen holder 107 and the subject.

Step (1): The subject is presented with the specimen 302 mounted to the face 202 for a time section t1.

Step (2): The ultrasonic motor 106 is rotated 90 degrees during the time section t2 so that the face presented to the subject changes from the face 202 to the face 201.

Step (3): The subject is presented with the specimen 301 mounted to the face 201 for a time section t3.

Step (4): The ultrasonic motor 106 is rotated 90 degrees in reverse to the direction of rotation in Step (2) during the time section t4 so that the face presented to the subject changes from the face 201 to the face 202.

Step (5): The subject is presented with the specimen 302 mounted to the face 202 for a time section t5.

With Steps (2) through (5) as one loop in the order of Step (2), Step (3), Step (4), Step (5), and so on, driving of the ultrasonic motor 106 and presenting of the specimens 301 and 302 is repeated N times, i.e., N loops.

Step (6): Finally, the specimen 302 mounted to the face 202 is presented for a time t6, and the flow ends. Note that the light quantity of illuminating the specimens 301 and 302 is constant with this example.

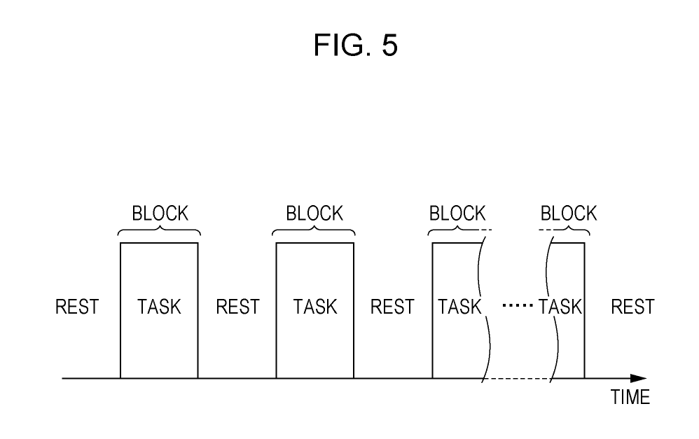

Generally, with fMRI brain function measurement, multiple presentation methods of stimulation to the subject are used, but with the present embodiment a technique called “block design”, which is commonly used in brain function measurement, will be used. Specifically, as shown in FIG. 5, a state called a “rest block” in which no sensory stimulation is applied to the subject, and a state called a “task block” in which sensory stimulation is applied to the subject, are alternately repeated.

Two types of images, i.e., a brain function image obtained in the rest period (an image in the state with no stimulation of the brain), and a brain function image obtained in the task period (an image in the state with stimulation of the brain), are obtained. By comparing the two images obtaining in this way, a brain function image reflecting the state of activity of the brain being subjected to sensory stimulation is obtained. In the present embodiment, presenting of the arbitrary shape of the specimen 301 corresponds to “task”, and presenting of the specimen 302 uniformly colored gray corresponds to “rest”.

The visual stimulation presentation sequence according to the present embodiment shown in FIGS. 4A through 4C, and the block design sequence shown in FIG. 5, are correlated as follows. Specifically, the step of presenting the specimen 301 to the subject for the duration of t3 in Step (3) corresponds to “task”, and the step of presenting the specimen 302 to the subject for the duration of t1, t5, and t6, in Steps (1), (5), and (6), corresponds to “rest”.

Next, the MRI brain function image acquisition sequence will be described with reference to FIG. 4C. Synchronously with the timing of presenting the visual stimulation to the subject, a brain function image at the time of presenting the arbitrary shape as the visual stimulation in time section t3 in Step (3) which is the task period, and a brain function image at the time of time section t5 in Step (5) which is the rest period, are each acquired. With the present embodiment, the Steps (2) through (5) are repeated N times, so N each are obtained of the brain function image under task and the brain function image in rest.

These are subjected to image processing such as averaging and so forth, so as to remove noise, and finally the images under task and in rest are compared, whereby a brain function image under task, i.e., when being presented with the arbitrary visual stimulation, can be obtained. Note that in Steps (1) and (6), brain function images of the subject in the rest state before the task-presenting experiment and after the experiment can be obtained, and thus the state of the brain functions at rest before the experiment of N loops of task and rest, and afterwards, can be compared.

Now, in the brain function image acquisition sequence according to the present embodiment, the ultrasonic motor 106 is rotated in Steps (2) and (4) to change the specimen being presented to the subject. It should be noted that the MRI brain function image acquisition sequence is preferably arranged such that no brain function image is obtained during the time sections of t2 and t4, for two reasons.

One reason is that the motion or rotation of the actual object with the specimen presentation apparatus according to the present embodiment itself changes the stimulation applied to the subject. Accordingly, a longer time is necessary to change the stimulation in comparison with a visual stimulation presentation experiment using a conventional display or projector, and as a result the visual stimulation of task and rest may be coexistent in this time section.

A second reason is that driving of the ultrasonic motor 106 generates faint electromagnetic waves, which may lead to deterioration in the brain function image. Stopping acquisition of brain function images during the time sections in which the ultrasonic motor 106 is being driven allows reduction of noise due to driving of the ultrasonic motor 106 from being superimposed on the image data to be used for analysis after measurement.

Now, with some MRI apparatuses, acquisition of brain function images cannot be stopped for just the time periods t2 and t4 as described above. In such cases, desired brain function images can be obtained by acquiring brain function images for the entire measurement period, but just performing analysis with the image data acquired in t3 and t5, without using the brain function images acquired in time sections t2 and t4. In this case as well, the brain function images obtained in t1 and t6 can be used for telling the state of the brain functions of the subject before and after measurement.

While the present embodiment has been described above as being configured using a specimen presentation apparatus to present the subject with visual stimulation and to perform brain function measurement thereof, the present invention is not restricted to such a configuration, and various modifications may be made to the configuration of the specimen presentation apparatus as described next.

For example, the specimen holder 107 is not restricted to a rectangular parallelepiped form, and any prismatic column which has specimen presentation faces parallel to a center axis of rotation, such as a triangular prism, pentagonal prism, hexagonal prism, or the like, may be used. Also, a rectangular flat plate having a center axis of rotation may be used as a specimen holder. It is preferable that at the time of presenting one face of the specimen holder to the subject, the other face is not visible from the subject.

Also, with the present embodiment, one each of printed sheets of paper serving as specimens are set to each of two adjacent faces of the specimen holder. However, multiple specimens may be set on each face, by changing the block design to be used with the brain function measurement. Also, specimens may be set to the four faces of the rectangular parallelepiped form specimen holder according to the present embodiment, using each of the faces 201, 202, 203, and 204 shown in FIG. 2A.

Moreover, the center axis of the specimen holder and the rotational shaft of the ultrasonic motor do not necessarily have to be on the same line.

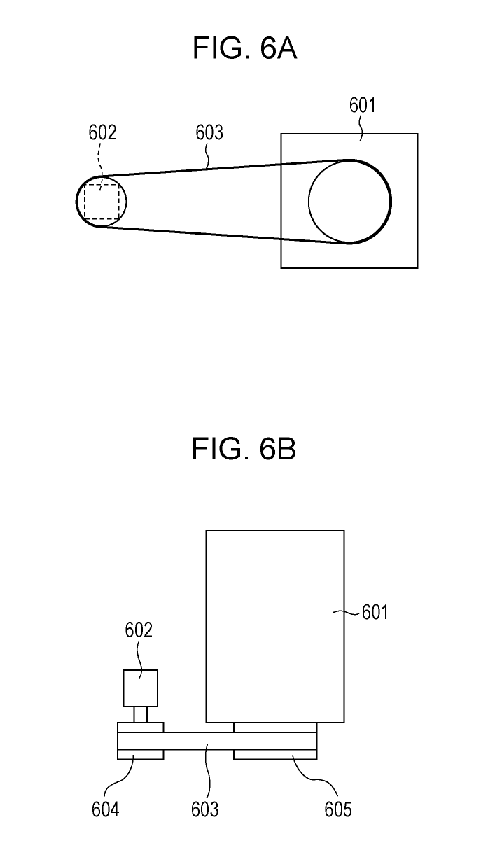

As shown in FIGS. 6A and 6B, of which FIG. 6A is a side view and FIG. 6B is a plan view, a configuration may be made wherein a specimen holder 601 and ultrasonic motor 602 are disposed with distance therebetween, connected by a rotational belt 603, so that the specimen holder is rotated. In this case, changing the ratio of diameter of pulley mechanisms 604 and 605 used to mount the belt to the specimen holder 601 and ultrasonic motor 602 allows the rotational speed of the specimen holder to be speed up, and also the resolution of angle control to be made finer.

Also, while an article obtained by printing on paper is used with the present embodiment, actual objects such as paintings, cloth, leather, and so forth, may be set in the specimen holder. Further, by improvising the specimen holder, thick and bulky objects may be used within the range of satisfying the geometric restrictions regarding the size within the bore of the MRI apparatus and the distance between the head of the subject and the specimen. Of course, these specimens do not contain magnetic substances, either.

### Second Embodiment

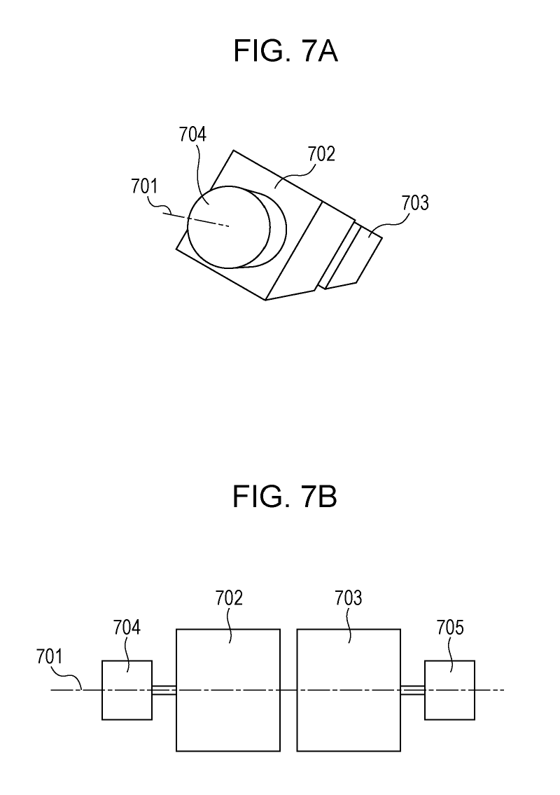

With the second embodiment, a configuration example wherein multiple specimen holders and ultrasonic motors are provided to the visual stimulation presentation apparatus, will be described with reference to FIGS. 7A and 7B, of which FIG. 7A is a perspective view and FIG. 7B is a plan view. With the specimen holders of the visual stimulation presentation apparatus according to the present embodiment, as shown in FIGS. 7A and 7B, two specimen holders 702 and 703 disposed on the same axis of rotation 701 are configured so as to be rotated by two ultrasonic motors 704 and 705. In this case, the specimen holder 702 is controlled by the ultrasonic motor 704 and the specimen holder 703 is controlled by the ultrasonic motor 705, respectively. Accordingly, brain function experiments can be performed wherein different types of visual stimulation are mounted to the specimen holders 702 and 703, and the brain activity is measured with the comparison of the two sets of specimens as the task.

### Third Embodiment

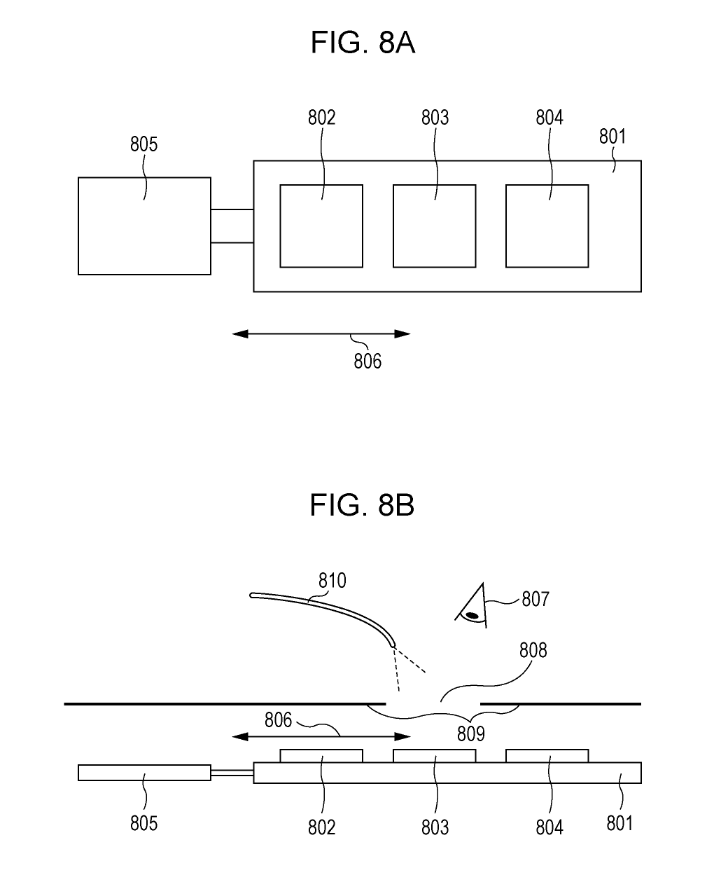

With the third embodiment, a configuration example wherein a linearly-driven actuator is used as the actuator for driving the specimen holder, rather than the rotational actuator configured of an ultrasonic motor as with the first embodiment, will be described with reference to FIGS. 8A and 8B. As shown in FIGS. 8A and 8B, multiple specimens 802, 803, and 804 are set in one row on the face of a specimen holder 801, with a linearly-driven actuator 805 being connected to the specimen holder 801. The linearly-driven actuator 805 moves the specimen holder 801 in the direction indicated by the arrow 806.

A shielding plate 809 having an observation window 808 is placed between the subject's eye 807 and the specimen holder 801, so that just one of the multiple specimens 802, 803, and 804 is presented to the subject. Also, illumination light is cast on the multiple specimens 802, 803, and 804 on the specimen holder 801 from an optical fiber illumination apparatus 810. The linearly-driven actuator 805 is driven to change the relative position between the specimen holder 801 and the observation window 808, thereby changing the specimen being presented to the subject. A linearly-driven actuator employing a piezoelectric device made of a non-magnetic material is preferable for the linearly-driven actuator 805.

### Fourth Embodiment

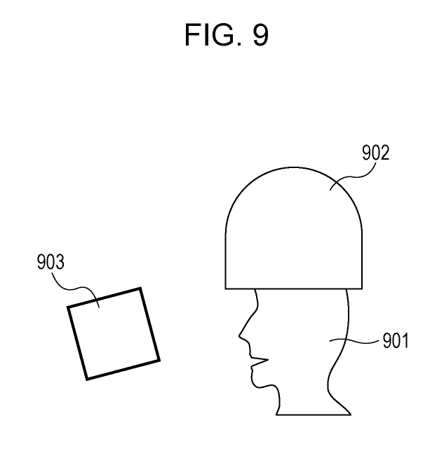

With the fourth embodiment, a configuration example of the visual stimulation presentation apparatus according to the present invention applied to magnetoencephalography (MEG) will be described with reference to FIG. 9. Using a magnetoencephalograph apparatus is desirable, since active portions of the brain in manifestation of brain functions can be measured and imaged with high time precision.

A magnetoencephalograph apparatus main unit (not shown) and visual stimulation apparatus for brain function measurement are set in a magnetically shielding room. A subject 901 wears a helmet-shaped Dewar sensor assembly 902 in which magnetic sensors are stored is mounted on the head. A specimen holder 903 of the visual stimulation apparatus for brain function measurement is set in front of the eyes of the subject 901.

The same procedures as those used with the brain function measurement method by functional magnetic resonance imaging according to the first embodiment may be used as the procedures for brain function measurement using this system. Brain function measurement can be performed with less noise for the magnetoencephalograph apparatus by using the visual stimulation apparatus for brain function measurement according to the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-123819 filed May 31, 2010, which is hereby incorporated by reference herein in its entirety.

What is claimed is:

1. A visual stimulation presentation apparatus used in presenting a subject with visual stimulation when running an apparatus to measure brain functions of the subject by detecting change in magnetic field, said visual stimulation presentation apparatus comprising:
a specimen holding unit, made of a non-magnetic material, configured to hold multiple specimens of actual objects as specimens for presenting the subject with visual stimulation;
an actuator, made of a non-magnetic material, configured to selectively present multiple specimens of actual objects set in said specimen holding unit to said subject, by driving said specimen holding unit; and
a control unit configured to control driving of said actuator.

2. The visual stimulation presentation apparatus according to claim 1, wherein said actuator is configured so as to be capable of being controlled when selectively presenting multiple visual stimulation specimens of actual objects to the subject such that said specimen holding unit is driven to a predetermined presentation position for a predetermined presentation time.

3. The visual stimulation presentation apparatus according to claim 1, wherein said actuator is configured of an actuator using a piezoelectric device.

4. The visual stimulation presentation apparatus according to claim 3, wherein said actuator using a piezoelectric device is an ultrasonic motor.

5. The visual stimulation presentation apparatus according to claim 3, wherein said actuator using a piezoelectric device is a linearly-driven actuator which linearly moves said specimen holding unit.

6. The visual stimulation presentation apparatus according to claim 1, further comprising:
an illumination unit configured to cast illumination light from an optional direction onto said specimens for visual stimulation which are selectively presented to said subject.

7. The visual stimulation presentation apparatus according to claim 1, wherein a plurality of said specimen holding units and a plurality of said actuators are included.

8. A functional magnetic resonance imaging apparatus including the visual stimulation presentation apparatus according to claim 1.

9. A magnetoencephalograph apparatus including the visual stimulation presentation apparatus according to claim 1.

10. A brain function measurement method using a visual stimulation presentation apparatus which selectively presents a subject with a plurality of visual stimulation specimens, to measure brain functions of the subject by detecting change in magnetic field, said method comprising the steps of:
selectively presenting said subject with a plurality of specimens which are actual objects, by driving a specimen holding unit made of a non-magnetic material in which specimens which are actual objects to serve as said plurality of visual stimulation specimens are set, by an actuator made of a non-magnetic material;
acquiring images in a state in which no sensory stimulation is applied to the brain of the subject, and in a state in which sensory stimulation is applied to the brain of the subject, said states being alternately repeated; and
obtaining a brain function image reflecting the state of activity of the brain, by comparing images acquired in said acquiring in a state in which no sensory stimulation is applied to the brain of the subject with images acquired in said acquiring in a state in which sensory stimulation is applied to the brain of the subject.

11. The brain function measurement method according to claim 10, wherein acquisition of images is stopped while said actuator is being driven to selectively present said plurality of specimens to said subject.
